(19) United States
(12) Patent Application Publication
(10) Pub. No.: US 2009/0324778 A1
(43) Pub. Date: Dec. 31, 2009

(54) NOVEL PREPARATION OF PHOSPHODIESTERASE OF PLANT ORIGIN

(75) Inventors: Eric Oriol, Champigny sur Marne (FR); Nadia Kaid, Nanterre (FR)

Correspondence Address: YOUNG & THOMPSON, 209 Madison Street, Suite 500, Alexandria, VA, 22314, US

(73) Assignee: LESAFFRE ET COMPAGNIE, Paris (FR)

(21) Appl. No.: 12/440,818

(22) PCT Filed: Sep. 11, 2007
(86) PCT No.: PCT/FR07/51905

§ 371 Date: Mar. 11, 2009

(30) Foreign Application Priority Data
Sep. 12, 2006 (FR) 0653686

(51) Int. Cl. A23L 1/28; C12N 1/38; C12P 19/34

(52) U.S. Cl. 426/60; 435/244; 426/62; 435/91.1

(57) ABSTRACT

The present invention relates to the preparation of a 5′-phosphodiesterase extracted from sorghum, and to its use for the preparation of a composition rich in 5′-nucleotides, in particular of a yeast extract.

The present invention relates to a method for preparing a flavour modifier by enzymatic treatment of yeast, the flavour modifier obtained and its uses.

Yeast extracts are commercially available in powdered or paste form, and are widely used in the agri-foodstuffs industry as flavouring agent.

The yeast extracts are defined as concentrates of the soluble fraction of yeasts which are generally obtained by autolysis or hydrolysis.

Autolysis is essentially a method aimed at damaging the plasma membrane of the yeast carried out by activating the actual degradative enzymes of the yeasts (proteases) and resulting in solubilization of the intracellular compounds.

Yeast extracts may also be obtained by hydrolysis using reagents or exogenous enzymes which allow the release of the intracellular content in a highly degraded form.

Yeast extracts have been widely accepted in the agri-foodstuffs industry under the name of natural flavouring agents.

They are also very competitive at the financial level, compared with other flavouring agents (which are often non-natural), with an equivalent flavour intensity.

These enzymatic treatments of yeasts are well known in the state of the art.

Yeast autolysates, which are prepared by subjecting the yeast to degradation by its own endogenous enzymatic material, are well known as food additives.

The autolysis may be triggered by incubating the yeast cells at a high temperature, adding organic solvents, using an increasing concentration of NaCl and combining these various methods.

During autolysis, the main conversion taking place is the degradation of proteins to peptides and amino acids.

The autolysates obtained consequentially have a bitter flavour and a typical pronounced yeast note. Another disadvantage is that the autolysates mainly contain only 3′-ribonucleotides, which make no contribution in terms of flavour because the endogenous ribonucleases convert intracellular RNA to 3′-ribonucleotides.

In patent EP 0 354 610 B1 by Quest International B.V., a method for preparing a yeast extract is taught in which, first of all, the wall is degraded with exogenous proteolytic enzymes under anaerobic conditions, and then the RNA is degraded with exogenous enzymes under oxidizing conditions. This method makes it possible to obtain an autolysate rich in 5′-nucleotides.

The known taste compounds contained in yeast autolysates include amino acids, peptides, nucleic acids such as 5′-nucleotides, saccharides and organic acids. The 5′-nucleotide content is of great importance for the taste and the flavour. Such nucleic acids are used as crude compounds for flavouring food preparations, for example 5′-inosine monophosphate (5′-IMP) or 5′-guanosine monophosphate (5′-GMP), but also find applications in the production of pharmaceutical products.

The unami taste is considered as the fifth basic taste alongside the sweet, salty, bitter and acid tastes. 5′-Nucleotides having a high taste-enhancing power also produce the unami taste, and thus reinforce the monosodium glutamate (MSG) effect that is naturally present in yeast extracts.

The production of 5′-nucleotides requires the hydrolysis of crude RNA by means of a very specific enzyme, 5′-phosphodiesterase (5′-PDE). Cohn and Volkin (1953) were the first to demonstrate the presence of a 5′-phosphodiesterase activity in snake venom. Despite its very high efficiency, it cannot be used in the agri-foodstuffs industry for obvious reasons.

Other major sources of 5′-PDE include certain fungi such as Penicillium citrinum and certain Actinomyces such as Streptomyces aureus.

In fact, the 5′-PDE obtained from Penicillium citrinum is commercially available from Amano Enzyme, among others, under the name Nuclease RP-1G (or EC3.1.30.1). Because of its very high production cost, this enzyme is mainly used on an industrial scale in an immobilized form. Its use in a method for producing yeast extracts enriched in 5′-nucleotides generates very high additional enzyme costs.

The presence of a 5′-PDE activity in certain plant germs has been demonstrated by Schuster (1957).

Numerous later studies were the subject of patent applications and mainly describe methods for producing yeast extracts rich in 5′-nucleotides from extracts of malt rootlets (malt being barley germ). Malt is a by-product of the brewery industry and may consequently therefore represent a cheap source of enzyme for the production of 5′-nucleotides from yeast RNA. In this case, the 5′-PDE is prepared by mere decoction of powdered malt rootlets in an aqueous solution containing zinc acetate to stabilize the enzyme, and its use generates a small additional cost for the manufacture of yeast extract enriched in 5′-nucleotides produced.

Methods for producing yeast derivatives rich in 5′-nucleotides using barley malt as source of enzymes are for example described in the documents U.S. Pat. No. 4,810,509, EP-A-0299078, FR 75 08446 and in the reference manual “Yeast Technology” by G. Reed and T. W. Nagodawithana, 2nd edition (Van Nostrand Reinhold, ISBN 0-442-31892-8), pages 382 to 385.

The main disadvantage of using 5′-PDE obtained from malt or barley germ is that it has an allergenic character. This is because the raw material from which the enzyme is derived, barley in this case, contains hordein, a prolamin similar to wheat gliadin which exhibits an allergenic character and causes intolerance (coeliac disease). In fact, barley is listed in the EC directive 2000/13/EC, amended by the EC directive 2003/89/EC, as belonging to cereals containing gluten. Thus, according to this directive, termed the “allergens” directive, products containing barley or its derivatives are subject to labelling.

The main characteristic of these proteins is that they are not degraded by the acidic environment of the stomach or by the intestinal digestive enzymes. Having remained intact, these proteins are absorbed as they are by the intestine and can thus trigger an immune reaction. In the case of coeliac disease, it is the consumption of these prolamins which causes a reaction in the body. All these prolamin proteins are toxic for so-called coeliac persons because their consumption causes a strong inflammatory reaction which damages the surface of the cells of the small intestine. This has the effect of reducing their capacity to absorb nutrients such as proteins, fats, carbohydrates, vitamins and minerals. The allergy is associated with symptoms of a gastrointestinal nature: stomach cramps, distension and chronic diarrhoea. These may lead to the malabsorption of several nutrients (iron, calcium, folic acid), malnutrition (anaemia and weight loss), fatigue, bone pain, muscle cramps and irritability.

The purification of PDE from barley malt may constitute an alternative if the enzyme can be successfully separated from the barley prolamin. However, the studies published by Ai-Yu Wang et al. ((1993) Biochemistry and Molecular Biology International, pp 1095-1102. vol. 29, No. 6), or by Beluhan et al. ((2003) in Biotechnology Letters 25, pp 1099-1103) focused on characterizing purified 5′-PDE from barley malt without measuring the presence of barley prolamin in their preparations. This alternative purification appears expensive and does not dispense with an analytical dossier demonstrating the removal of gluten from the enzymatic preparation but especially with a clinical dossier proving the absence of an allergenic character of the product resulting from the action of this enzyme.

A second alternative consists in using a 5′-PDE of microbial origin obtained from strains of filamentous fungi such as Penicillium citrinum or Aspergillus niger, or from bacteria such as Actinomyces or Streptomyces. However, these are lengthy developments which involve strain improvement phases. In addition, this type of production requires industrial fermentation/purification plants and a process which in the end results in an expensive enzyme.

The third alternative would be to clone a plant 5′-PDE into an industrial microorganism. This alternative has been evaluated by the applicant's research teams but, besides the fact that the sequences encoding this enzyme are not known, this would lead to an enzyme derived from a genetically modified organism (GMO), a product type which is still very poorly viewed by various user clients.

Thus, the unsolved problem to date consists in finding a source of gluten-free food enzyme which does not have an economic disadvantage as is the case of the commercial enzymes currently available on the market. This preparation should moreover be free of nucleases or phosphatases which degrade RNA fragments or nucleotides and lead to degradation products with no taste-enhancing power. It should not produce tastes or flavour notes either which could be perceived as being awkward during tasting.

The applicant has just now discovered, particularly unexpectedly and surprisingly, that sorghum malt can perfectly replace barley malt having an allergenic character in the preparation of yeast extracts rich in 5′-nucleotides. What is more, sorghum is not on the list of gluten-containing cereals according to the directive C2003/89/EC, and consequently has no allergenic character like gluten.

The applicant has developed an enzymatic preparation of 5′-PDE from a mixture containing sorghum malt germs and rootlets.

More particularly, the sorghum 5′-phosphodiesterase is obtained by means of a decoction of malted sorghum rootlets. Thus, a powder of sorghum rootlets having a particle size between 20 and 2000 μm, preferably between 100 and 200 μm, is suspended in an aqueous solution of zinc acetate or of an equivalent agent, that is to say an agent having the same 5′-PDE stabilizing effect. The suspension is incubated at a temperature between 50 and 80° C. for 30 min to 4 h. Preferably, this decoction is produced with stirring. For example, the rootlet powder may be used in a proportion of 5 to 20% (w/w), preferably 10 to 15%, and in particular 13%. The concentration of zinc acetate is preferably between 0.2 and 5 g/l. In an alternative embodiment, a whole sorghum malt containing rootlets is used. Next, in a preferred embodiment, the soluble portion of the solution is recovered. For example, the preparation may be separated on a centrifugal decanter and then the supernatant is clarified in a centrifugal clarifier.

The sorghum 5′-PDE preparation thus obtained may be used for the preparation of a gluten-free composition rich in 5′-nucleotides, of a yeast extract rich in 5′-nucleotides.

The present invention therefore relates to a method for preparing a sorghum 5′-PDE comprising the preparation of a decoction of malted sorghum rootlets in an aqueous solution of zinc acetate (0.2 to 5 g/l) at a temperature of between 50 and 80° C. for 30 min to 4 h, and then the removal of the insoluble fraction. It may also comprise a concentration step.

The present invention relates to the preparation of sorghum 5′-PDE capable of being obtained by this method and its use to digest RNA, more particularly for the preparation of a composition rich in 5′-nucleotides, preferably a yeast extract, said composition being free of gluten. It also relates to the use of 5′-PDE extracted from sorghum malt to enrich an RNA-containing composition in 5′-nucleotides. It therefore relates to a method for preparing a gluten-free composition rich in 5′-nucleotides, preferably a yeast extract, comprising the treatment of microbial cells so as to release the RNA into the extracellular medium, and the treatment of the suspension obtained with a sorghum 5′-PDE preparation to convert the RNA released to 5′-nucleotides. Examples of other microorganisms which may be used in this method are for example a filamentous fungus of the Aspergillus or Trichoderma type or a bacterium, preferably a lactic acid bacterium of the genus Lactobacillus. The method may comprise an intermediate step of complete or partial purification of the RNA (see JP 51106791).

In a preferred embodiment, it comprises the treatment of a yeast extract or of a yeast suspension with a sorghum 5′-PDE preparation.

The present invention therefore relates more particularly to a method for preparing a yeast extract rich in 5′-nucleotides and free of gluten, comprising:
the heating of a yeast suspension;
the treatment of the yeast suspension with a 5′-phosphodiesterase;
the separation of the insoluble materials from the suspension; and
the recovery of the yeast extract;

characterized in that the 5′-phosphodiesterase is extracted from sorghum, in particular malted sorghum, more particularly from malted sorghum rootlets.

The yeast useful in the present invention is an edible yeast. According to the invention, the yeast used to prepare the extract preferably belongs to the genus Saccharomyces and preferably still belonging to the species Saccharomyces cerevisiae, including that called Saccharomyces carlsbergensis. The said Saccharomyces cerevisiae yeast cells are also often called Saccharomyces carlsbergensis in the case of brewer's yeast, the exact taxonomic name being Saccharomyces cerevisiae according to “THE YEASTS, a taxonomic study”, 3rd edition, published by N. J. W. Kreger van Rij—1984 (on the other hand, according to the 1998 4th edition of this manual, Saccharomyces carlsbergensis has two synonyms: Saccharomyces cerevisiae and Saccharomyces pastorianus, it is the 3rd edition of this manual dating from 1984 which is taken as reference in the present document). Moreover, the yeast may also be derived from the genus Candida (for example, C. utilis), Pichia, Hansenula, Kluyveromyces (for example K. lactis, K. marxanius or K. fragilis), Torula, Fusarium, Zymonomas and the like. The yeast may be derived from a culture of fresh yeasts or from yeasts which have been used in brewing processes. In a preferred embodiment, the yeast is of the genus Saccharomyces, of the genus Candida or of the genus Kluyveromyces.

The yeast will be chosen such that it has a high RNA content. In a particular embodiment, the RNA content is between 6 and 15% by dry weight.

The yeast may have been subjected to a preliminary treatment which makes it possible to increase its RNA content. Such treatments are described in U.S. Pat. No. 3,909,352 and JP 11-196856 by mutation and selection of the yeasts and in JP 5-176757 by limiting potassium sulphate in the culture medium.

The RNA content of the yeast suspension may be increased by adding RNA obtained from a microorganism authorized in human foodstuffs or in animal feedstuffs. Examples of such microorganisms are for example a filamentous fungus of the Aspergillus or Trichoderma type or a bacterium, preferably a lactic acid bacterium of the genus Lactobacillus. In this case, the microorganism is treated so as to release its RNA content. In a particular embodiment, the RNA may be completely or partially purified by known methods (ultrafiltration, chromatography or precipitation).

The methods for preparing a yeast extract are well known to a person skilled in the art. Such methods are for example described in the following patents: EP 249 435; EP 299 078; EP 354 610; EP 466 922; EP 1 199 353; EP 1 479 299; U.S. Pat. No. 3,961,080; U.S. Pat. No. 4,303,680; U.S. Pat. No. 4,810,509. These methods generally comprise a step for heating a yeast suspension, optionally a step for autolysis and/or hydrolysis of the yeast, and preferably a step for separating the insoluble materials from the suspension. The yeast suspension is a suspension of live yeasts.

Preferably, the yeast suspension is plasmolysed so as to inactivate the yeast enzymes, including phosphatases and nucleases, permeabilize the yeast so that it releases its cellular content and its RNA, and preferably selectively solubilize the RNA in the extracellular medium, which in the end makes it possible to obtain 5′-nucleotide titres greater than the RNA content of the yeast suspension. Preferably, the yeast suspension comprises between 10 and 25% dry matter. In a preferred embodiment, the yeast suspension is heated for 5 min to 3 h at a temperature ranging from 5 to 95° C. In particular, the suspension may be heated for 2 h at 75° C. and then cooled to 60° C.

It is also possible to use other techniques well known to a person skilled in the art to release the cellular content of the yeast, for example by a mechanical treatment (French press, glass beads, pulsed electromagnetic field, and the like), by chemical treatment (acids, bases, salts, solvents, detergents, and the like) or by enzymatic treatment (β-glucanases, chitinases, proteases, and the like).

Moreover, the method may also comprise an enzymatic digestion step, it being possible for the enzymes to be chosen from proteases, β-glucanases, amylases, lipases and the like.

Thus, the present invention relates to a method for preparing a yeast extract rich in 5′-nucleotides, comprising:
a) the heating of a yeast suspension;
b) the autolysis and/or enzymatic hydrolysis of the yeast;
c) the separation of the insoluble materials from the suspension; and
d) the recovery of the yeast extract;
   characterized in that the method comprises the treatment of the yeast suspension with a 5′-phosphodiesterase extracted from sorghum malt.

Step b is preferably performed using enzymes exogenous to the yeast, in particular proteases in order to increase the degradation of the yeast proteins. Examples of such proteases are plant proteases (papain, bromelain and the like) or microbial proteases (Bacillus subtilis, Aspergillus oryzae, and the like).

The method may comprise a step for the complete or partial purification of the RNA before the step for treatment with the sorghum 5′-phosphodiesterase.

In a preferred embodiment, the step for treating the yeast suspension with the sorghum 5′-PDE is preferably carried out at a pH of between 5.0 and 7.5 with a temperature of between 35 and 70° C. The time for incubation of the yeast suspension with the sorghum 5′-PDE may vary from 5 to 30 h. Preferably, the sorghum 5′-PDE preparation is added to the yeast suspension in an amount of 10% w/w. For example, the step for treating the yeast suspension with the sorghum 5′-PDE may be performed at 60° C. for 18 h at a pH of 6.3.

Preferably, the yeast extract is rich in 5′-GMP and/or 5′-IMP. In particular, 5′-GMP and 5′-IMP levels of 0.1 to 15% each (expressed in the form of the disodium and heptahydrate salt) may be achieved, preferably 2 to 5%. The yeast extract obtained is free of gluten.

The method may additionally comprise a step for treating the yeast suspension with a deaminase. This treatment may be performed during or after treating the yeast suspension with the sorghum PDE. In a preferred embodiment, this step for treating with a deaminase is carried out simultaneously with or after the step for treating with the sorghum PDE. For example, the deaminase may be added during the final hours of the treatment with 5′-PDE, after having cooled the suspension, for example to 45° C. This step makes it possible to convert the 5′-AMP to the desired 5′-IMP. This additional step is well known to a person skilled in the art and is described for example in EP 249 435 and EP 354 610. One example of a commercially available deaminase is Deamizyme 50000G produced by Amano. In a particular embodiment, the method comprises a fermentation step which makes it possible to convert the polysaccharides to organic acids such as lactic or succinic acid. This step is well known to persons skilled in the art and is for example described in EP 191 513 and EP 354 610. This fermentation may be preferably carried out with the aid of bacteria of the genus Lactobacillus or equivalent.

Preferably, the yeast extract is then separated from the insoluble portion of the yeast cells. The yeast extract thus separated from the insoluble portion offers the advantage of a better preservation without the appearance of flavour notes due to the oxidation of the membrane lipids of the insoluble portion. For example, this step may be carried out by centrifugation or filtration or recovery of the liquid fraction.

The liquid fraction may then be subjected to all the downstream treatments known for yeast extracts, and in particular concentration, filtration, pasteurization and/or drying.

As indicated above, yeast extracts are commonly used as taste enhancer. The expression “yeast extract” is understood to mean, according to the invention, concentrates of the soluble fraction of yeasts, generally obtained by autolysis or hydrolysis.

The yeast extract rich in 5′-nucleotides may be provided in various forms, such as, for example, in liquid, pasty or solid form. The yeast extract advantageously comprises at least 90% by mass, preferably between 94 and 98% by mass, of dry matter.

The invention therefore relates to the yeast extracts obtained or capable of being obtained by the method according to the present invention or their applications in the food sector. These yeast extracts are free of gluten. They preferably comprise 5′-GMP and 5′-IMP levels of 0.1 to 15% each (expressed in the form of the disodium or heptahydrate salt).

The present invention relates to the use of the yeast extracts according to the present invention in a dietary formula, the dietary formula thus obtained and the food product for consumption obtained based on this formula. The dietary formula may for example be a broth, soup, sauce, prepared meal, baker's dough or condiment formula.

The invention also relates to flavour modifiers comprising a yeast extract according to the present invention. The flavour modifier according to the present invention may also comprise insoluble materials.

The invention also relates to the use of a flavour modifier according to the present invention in a dietary formula, the dietary formula thus obtained and the food product for consumption obtained based on this formula. The dietary formula may for example be a broth, soup, sauce, prepared meal, bakery product or condiment formula.

Thus, the invention relates to a method for flavouring food products, characterized in that a flavour modifier according to the present invention is used.

The examples below are provided in order to illustrate the invention and should in no way be considered as limiting the scope of the invention.

### EXAMPLE 1

### Preparation of the Extract of Sorghum Rootlets

The rootlets used are derived from an African sorghum malt factory. 13 g of sorghum rootlets are ground or milled so as to obtain a powder having a mean particle size of 500 μm. This powder is made into a decoction at 13% (w/w), in an aqueous solution of zinc acetate at 0.2 g/l, at a temperature of 60° C., for 2 hours. The extract of sorghum rootlets is obtained after extraction of the solids and insolubles from the decoction by two centrifugal separation steps.

### EXAMPLE 2

### Preparation of a Yeast Extract Rich in 5′-Nucleotides—Comparison Between the 5′-PDE Derived from Sorghum and the Commercial Enzyme

1000 g of cream yeast of the genus Saccharomyces cerevisiae having a dry matter content of 15% and an RNA content of 9.5% (measured by hydrolysis according to the Trevelyan method as described in: Induction of Autolytic Breakdown of RNA in Yeast by Addition of Ethanol and by Drying/Rehydration; Trevelyan, J., Sci. Food Agric., 1977, 28, 579-588; and in Processing Yeast to Reduce its Nucleic Acid Content. Induction of Intracellular RNAse Action by a Simple Heat-shock Procedure, and an Efficient Chemical Method Based on Extraction of RNA by Salt Solutions at low pH; Trevelyan, J., Soc. Chem. Ind., 1978, pp 141-174) are heat-treated at 75° C. for 2 hours, and then cooled to 56° C. and adjusted to a pH of 5.3 (plasmolysis).

This cream is mixed with the supernatant of the decoction of sorghum rootlets containing 5′-PDE (10% w/w). The whole is left to incubate with gentle stirring for 15 h at 56° C. so as to hydrolyse the RNA into 5′-nucleotides. The solids are then separated by centrifugation and subjected to washing with demineralized water before being again centrifuged. The two supernatants are combined, concentrated and spray-dried. Analysis of the powder shows a 5′-GMP content of 2.44% (expressed in the form of the disodium and heptahydrate salt). The assay is performed by RP-HPLC, as described in: Enzymatic Production of Ribonucleotides from Autolysates of Kluyveromyces marxianus grown on whey; Belem, M. A. F., et al., Journal of Food Science, 62, pp 851-854.

1 kg of the same cream, plasmolysed under identical conditions and incubated for 15 h with 1 g of commercial 5′-PDE from Amano (trade name=PDE RP-1, Amano Enzyme Europe, Ltd) gives, after separation, concentration and drying, a powder having a titre of 2.60% 5′-GMP. The efficiency of hydrolysis is therefore similar in both cases, and the 5′-PDE derived from sorghum is equally efficient in terms of hydrolysis of RNA as the much more expensive commercial enzyme.

### EXAMPLE 3

The method is carried out starting with 13 g of malted sorghum rootlets and 1000 g of cream yeast as described in the preceding examples. However, after 15 h of incubation, the temperature is reduced to 45° C. and 0.1 g of 5′-adenylic deaminase from Amano (trade name=Deamizyme 50000G, Amano Enzyme Europe, Ltd) is added. The whole is incubated for 5 h in order to allow the conversion of 5′-AMP to 5′-IMP by the 5′-adenylic deaminase. The solids are then separated by centrifugation and subjected to washing with demineralized water before being again centrifuged. The two supernatants are combined, concentrated and spray-dried. Analysis of the powder shows a 5′-GMP content of 2.80% and a 5′-IMP content of 3.21% (assay by RP-HPLC, both expressed in the form of the disodium and heptahydrate salt).

### EXAMPLE 4

The method is carried out starting with 13 g of malted sorghum rootlets and 1000 g of cream yeast as described in the preceding examples. However, before the addition of the sorghum malt 5′-PDE, 0.5 g of papain is added in order to increase the solubilization of the dry matter content of the yeast. The remainder of the method is identical, including as in Example 3, the addition of 5′-adenylic deaminase. Analysis of the resulting powder shows a 5′-GMP content of 1.96% and a 5′-IMP content of 2.07% (assay by RP-HPLC, both expressed in the form of the disodium and heptahydrate salt).

### EXAMPLE 5

The method is carried out as in Examples 1 and 2, except that the Saccharomyces cerevisiae cream yeast is replaced with a Candida utilis cream having a dry matter content of 13% and a titre of 12.5% RNA. Analysis of the resulting powder shows a 5′-GMP content of 3.75% and a 5′-IMP content of 3.98% (both expressed in the form of the disodium and heptahydrate salt).

1-14. (canceled)

15. Method for preparing a yeast extract rich in 5′-nucleotides and free of gluten, comprising:
a) heating of a yeast suspension;
b) treatment of the yeast suspension with a 5′-phosphodiesterase;
c) separation of the insoluble materials from the suspension; and
d) recovery of the yeast extract; wherein the 5′-phosphodiesterase is extracted from sorghum malt rootlets.

16. Method according to claim 15, wherein the 5′-phosphodiesterase extracted from sorghum malt rootlets is prepared by decoction of a powder of sorghum rootlets in suspension in an aqueous solution of zinc acetate (0.2 to 5 g/l) at a temperature between 50 and 80° C. for 30 min to 4 h and by removing the insoluble fraction.

17. Method according to claim 16, wherein the particle size of the powder is between 20 and 2000 μm.

18. Method according to claim 17, wherein the particle size of the powder is between 100 and 200 μm.

19. Method according to claim 15, wherein the step for treating the yeast suspension with the sorghum 5′-phosphodiesterase is carried out at a pH of between 5.0 and 7.5 with a temperature of between 35 and 70° C. and for 5 to 30 h.

20. Method according to claim 15, wherein the method additionally comprises a step for enzymatic digestion of the yeast suspension after the heating step.

21. Method according to claim 15, wherein the method additionally comprises a step for the complete or partial purification of the yeast RNA before the step for treatment with the sorghum 5′-phosphodiesterase.

22. Method according to claim 15, wherein the method additionally comprises a step for treating with a deaminase.

23. Method according to claim 15, wherein the RNA content of the yeast is between 6 and 15% by dry weight.

24. Method according to claim 15, wherein the yeast is of the genus Saccharomyces, of the genus Candida or of the genus Kluyveromyces.

25. Yeast extract rich in 5′-nucleotides and free of gluten obtainable by the method according to claim 15.

26. Flavour modifier comprising a yeast extract rich in 5′-nucleotides and free of gluten obtainable by the method according to claim 15.

27. Method for flavouring food products, wherein a flavour modifier according to claim 26 is used.

28. Food product comprising a flavour modifier according to claim 26.

29. Method for the preparation of a composition rich in 5′-nucleotides, by using a 5′-phosphodiesterase extracted from sorghum malt.
